(19) United States
(12) Patent Application Publication
(10) Pub. No.: US 2011/0014260 A1
(43) Pub. Date: Jan. 20, 2011

(54) SYSTEM AND METHOD FOR LIVER CELL CULTURE AND MATURATION

(76) Inventors: Eric Novik, Edison, NJ (US); Martin L. Yarmush, Newtown, MA (US); Rene Schloss, East Brunswick, NJ (US); Nripen Sharma, Cambridge, MA (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/677,248

(22) PCT Filed: Sep. 11, 2008
(86) PCT No.: PCT/US08/76014

§ 371 Date: Oct. 1, 2010

Related U.S. Application Data
(60) Provisional application No. 60/993,372, filed on Sep. 11, 2007.

(51) Int. Cl. A61K 9/00; C12N 5/071; A61K 35/12; A61P 43/00

(52) U.S. Cl. 424/422; 435/325; 435/366; 435/354; 424/93.7

(57) ABSTRACT

The present invention relates to systems and methods for maturation, proliferation and maintenance of function in cells presenting hepatocyte characteristics and differentiated from stem cells. The cells of the present invention may be generated from stem cell grown in collagen sandwich configuration in the presence of a morphogen (e.g. S-NitrosoAcetylPenicillamine (SNAP) or Oncostatin-M (OSM)).

### CROSS REFERENCE TO RELATED PATENT APPLICATIONS

The present application claims priority from Provisional U.S. Patent Application Ser. No. 60/993,372, which was filed on Sep. 11, 2007.

### FIELD OF THE INVENTION

The present invention relates to systems and methods for maturation, proliferation and maintenance of function in hepatocytes differentiated from stem cells.

### BACKGROUND OF THE INVENTION

Acute liver failure affects hundreds of thousands of people per year around the globe and in many cases is resolved with an orthotopic liver transplant. Due to a shortage of donor organs many patients will die while waiting for a donor organ to become available. Extracorporeal liver assist devices (LAD) could help to bridge patients to transplant however, this technology is limited by a lack of an adequate hepatocyte cell source (Tilles et al. 2002a; Tilles et al. 2002b). Pluripotent embryonic stem cells (ES) represent a promising renewable cell source to generate hepatocyte lineage cells, which have been incorporated into implantable engineered tissue constructs (Soto-Gutierrez et al. 2006) and ex vivo cell based therapeutic devices such as LADs (Cheul H. Cho et al. 2007). However, current differentiation techniques have not yet generated the large and functionally sustainable cell masses which would be required to make such therapies clinically available.

ES differentiation into hepatocyte lineage cells, using a variety of differentiation platforms such as monolayer (Sharma et al. 2006), encapsulation (Maguire et al. 2006) and EB mediated (Hamazaki et al. 2001; Heo et al. 2006; Kumashiro et al. 2005b), have been previously described by many investigators. Of these, EB mediated differentiation, which mimics in vivo embryogenesis, has been characterized most completely. For example, following exogenous growth factor supplementation and co-culture with nonparenchymal liver cell lines, investigators have demonstrated EB mediated differentiation yields up to a 70% albumin-positive population, which expresses a variety of liver lineage genes and metabolizes lidocane and diazepam (Soto-Gutierrez et al. 2007). Additionally, in vitro aggregation of murine ES cells initiates the formation of EBs which has been shown to facilitate spontaneous differentiation in the absence of growth factor and extracellular matrix supplementation, resulting in liver lineage cells characterized by 80% albumin expression as well as mature hepatocyte genes such as Cytochrome P450 detoxifying enzymes (CYP450) (Novik et al. 2006; Tsutsui et al. 2006).

As noted above, the majority of these previously reported studies, using western blot analysis and RT-PCR, have shown gene expression profiles for a variety of genes most commonly associated with liver differentiation such as, albumin and alfa-fetoprotein and have used ALB-GFP promoters to isolate hepatocyte lineage cells. Although most include growth factors, it has also been shown that hepatocyte differentiation can occur spontaneously, without stimulus from exogenous growth factors. To this end, there is almost no consensus on which platform to use to differentiate hepatocyte lineage cells from ES cells. Techniques range from encapsulation to co-culture and no two platforms are alike. The cells produced by these different culture methods express similar genetic mRNA, are phenotypically similar and are similar to recent studies which have included albumin secretion, urea secretion and CYP7a1 expression, which has been shown to be hepatocyte specific. However, few of these studies illustrate detoxification mediated by specific CYPs, which is critical for their use in BAL treatment, drug discovery studies, or for implantation. Another point that has gone largely unresolved is the long term propagation of differentiated cells while still maintaining differentiated function. While some studies investigated the effects of Oncostatin-M (OSM) and sodium butyrate, a nitric oxide (NO) donor, on fetal liver hepatocytes and have shown that their supplementation maintains long term structure and function as well as inducing further differentiation (Ehashi et al. 2005; Iwai et al. 2002), at present, these are the only compounds studied thus far and each of these have been shown to be limited in their ability to yield hepatocyte cells with normal to high hepatocytic activity levels. Since propagation is essential for scale-up and will play an important role in generating the large cell mass required from the small number of hepatocyte precursors isolated from the whole population, it is desirable to have a system and method for producing such hepatocyte cell populations that may be replicated with relative ease.

Based on the foregoing, a system and method is desirable for producing and isolating mature hepatocytic cells, which effectuate normal to high levels of hepatocytic activity and detoxification both during incubation and well after the incubation period. As set forth herein, the present invention addresses the forgoing needs.

### SUMMARY OF THE INVENTION

The present invention relates to systems and methods for maturation, proliferation and maintenance of function in hepatocytes differentiated from stem cells. More specifically, the present invention relates to liver lineage cells generated using stem cell differentiation systems and plating these differentiated cells onto a secondary culture (e.g. collagen sandwich configuration) that is supplemented with a morphogen (e.g. S-NitrosoAcetylPenicillamine (SNAP) or Oncostatin-M (OSM)). Such a supplemented secondary culture facilitates maturation and maintenance of liver function in embryonic stem cell-derived liver lineage cells. This technique is advantageous in that it allows for rapid proliferation of differentiated cells with retention of hepatic function for extended periods of time. Moreover, these cells exhibit improved hepatic function (e.g. protein expression, secretion, and detoxification) relative to previously reported results. The systems and methods of the present invention encompass such characteristics, thus enabling production of liver lineage cells with applications in bioartificial devices, environmental biosensors, and drug screening.

The need for a well characterized, homogeneous, sustainable, ES derived hepatocyte-like cell forms the basis for the present invention. The discussion and examples provided herein were designed to identify the differentiation condition which most effectively induces the differentiation of hepatocyte lineage cells. This population was then propagated in secondary culture in order to generate a large and functional cell mass. Based on the foregoing, it was discovered that the morphogens SNAP and OSM yielded ES derived hepatocytes that are homogeneous, are sustainable, and exhibit hepatic characteristic (e.g. marker protein expression, secretion, and detoxification). In a preferred embodiment, although not limited thereto, collagen sandwiches were used to augment and/or maintain these functions for extended periods of time.

As noted below, in a most preferred embodiment, differentiated stem cells are plated within a secondary culture, e.g. collagen sandwich configuration, and incubated in the presence of either SNAP or OSM for at least 10 days. Such steps and incubation time periods allowed for maintenance and augmentation of function of the differentiated hepatocyte-like cells, particularly spontaneously Embryoid Body (EB)-mediated hepatocytic cells. Such steps and incubation periods also yielded an increase in cell number (e.g. from 5×10⁴ Day 17 cells to 1×10⁶ cells within 10 days) over previous methods, while still maintaining 80% ALB expression. As discussed further below, in one embodiment at least 10% of the cells of the present invention exhibited positive hepatocyte-like activity (e.g. CK 18 expression) after six days, eight days, or ten days in the supplemented culture. In another embodiment at least 15% of the cells of the present invention exhibited positive hepatocyte-like activity (e.g. CK 18 expression) after six days, eight days, or ten days in the supplemented culture. In a further embodiment at least 20% of the cells of the present invention exhibited positive hepatocyte-like activity (e.g. CK 18 expression) after six days, eight days, or ten days in the supplemented culture. In even further embodiments, at least 30%-60% of the cells of the present invention exhibited positive hepatocyte-like activity (e.g. CK 18 expression) after ten days in the supplemented culture. In an alternative embodiment, the cells of the present invention are also characterized by secretion of albumin in an amount between 40 ng per 10⁶ cells per day and 70 ng per 10⁶ cells per day after about 10 days in the supplemented secondary culture. In another embodiment, the cells of the present invention are characterized by secretion of urea in an amount of at least 15 ng per 10⁶ cells per day after about 10 days in the supplemented secondary culture. In a further embodiment, the cells of the present invention, after being cultured for at least about 10 days in supplemented secondary media, are characterized by having cytochrome P450 activity corresponding to at least about 200 uM/ml resorufine after 30 minutes.

As discussed further herein, detoxification was also detected in the cells and cell populations of the present invention. More specifically, the hepatocyte-like cells of the present invention presented detoxification after incubation and within the Sandwich/morphogen condition. In a most preferred embodiment, the detoxification was via CYP450 metabolism during and post incubation. While Xenobiotic metabolism has been well characterized in primary hepatocyte systems and, although there have been reports of induction of CYP450 mRNA in ES derived hepatocyte-like cells, this aspect is advantageous to the present invention because few reports actually provide for sustainable detoxification that is detectable post-incubation, as seen in the present invention.

The fact that cells isolated from primary EB culture can proliferate in the Sandwich/morphogen condition, while maintaining their hepatocyte-like characteristics, brings added value to generating the large mass of cells required for use in in vitro drug screening systems and liver assist devices. The present system affords a combination of maintenance and augmentation of hepatocyte specific functions in conjunction with an increase in cell mass in the Sandwich/morphogen condition for at least four weeks post differentiation induction.

The present invention, including the foregoing methods, systems and advantages has a plurality of potential uses. As discussed further herein, hepatocyte-like cell populations of the present invention may be used either in drug studies to test the effects and or metabolic breakdown of a prior or potential drug or to screen the effect of certain compounds on the cell types. Alternatively, the cells of the present invention may be used as a biological tool testing the biological effects of a particular compound on the drug. In other embodiments, the hepatocyte-like cell populations of the present invention may also be directly administered to a subject in need thereof wherein the cells are formulated in any conventional manner and are administered using one or more physiologically acceptable carriers, excipients and other auxiliaries. In other embodiments, the cells of the present invention may be administered as tissue constructs in cooperation with bioartificial liver support (BAL) such as extracorporeal liver assist devices (LAD). In accordance with the foregoing, tissue constructs, BALs, LADs, and other similar medical devices may be used in conjunction with the cell populations of the present invention in any conventional manner known by one of ordinary skill in the art.

### BRIEF DESCRIPTION OF THE DRAWINGS

FIG. 1. illustrates non supplemented secondary culture characterization. A) Cell number was assessed by counting total cells dissociated following trypsinization. Cells were plated into tertiary culture at 5×10⁴ cells per well at day 6 B) Time course of the percentage of cells expressing albumin in polystyrene secondary culture. Each data point represents the % of cells with an intensity reading above 0. The average of three experiments is presented. All values were statistically significant as compared to the ES control. C) Time course of the percentage of cells expressing CK18 in polystyrene secondary culture. Each data point represents the % of cells with a normalized intensity reading above 0. The average of three experiments is presented. All values were statistically significant as compared to the ES control.

FIG. 2. illustrates OSM and SNAP supplemented secondary culture characterization. A) Cell number was assessed by counting total cells dissociated following trypsinization. Cells from the three conditions were plated into tertiary culture at 5×10⁴ cells per well at day 6 B) Time course of the percentage of cells expressing albumin in polystyrene secondary culture. Each data point represents the % of cells with an intensity reading above 0. The average of three experiments is presented. All values were statistically significant as compared to the ES control. Asterisk (*) indicates statistically significant differences (p<0.05) from other conditions on that day. C) Time course of urea secretion rates in the supplemented conditions. The average of three experiments is presented. All values were statistically significant as compared to the ES control. Asterisk (*) indicates statistically significant differences (p<0.05) from other conditions on that day.

FIG. 3. illustrates collagen sandwich secondary culture characterization. A) Cell number was assessed by counting total cells dissociated following trypsinization. Cell number in all GEL conditions was assessed by counting the total number of cells dissociated following collagenase digestion and trypsonization. Asterisk (*) indicates time point at which the NS cells were passed to 5×10⁴ cells per well. B) Time course of the percentage of cells expressing albumin in polystyrene secondary culture. Each data point represents the % of cells with an intensity reading above 0. The average of three experiments is presented. All values were statistically significant as compared to the ES control. Asterisk (*) indicates statistically significant differences (p<0.05) from NS and GEL conditions on that day. C) Time course of the percentage of cells expressing CK18 in the sandwich culture conditions. Each data point represents the % of cells with an intensity reading above 0. The average of three experiments is presented. All values were statistically significant as compared to the ES control. Asterisk (*) indicates statistically significant differences (p<0.05) from all other conditions on that day.

FIG. 4. illustrates albumin and urea secretion rates in sandwich culture. A) Time course of ALB secretion rates in the sandwich culture conditions. The average of three experiments is presented. All values were statistically significant as compared to the ES control. Asterisk (*) indicates statistically significant differences (p<0.05) from all other conditions on that day. B) Time course of urea secretion rates in the sandwich culture conditions. The average of three experiments is presented. All values were statistically significant as compared to the ES control. Asterisk (*) indicates statistically significant differences (p<0.05) from all other conditions on that day.

FIG. 5. illustrates cellular morphologies. A) 10× magnification, phase contrast image of cells in the GEL condition. Cells with similar morphologies were observed in all Gel conditions B) 20× magnification, phase contrast image of cells described in A. C) 10× magnification, phase contrast image of GSNAP cells in a non confluent, loosely connected environment. D) 20× magnification, phase contrast image of GSNAP cells were greater than 95% of cells are in groups of round or square cells indicated by white arrows. e) 20× magnification, phase contrast image of NG cells. Cells with similar morphologies were observed in all non GEL conditions.

FIG. 6. illustrates cytochrome P450 detoxification. All graphs represent cells which have been in secondary culture for 10 days. Hepa 1-6 were used as a positive control. A) BROD activity is measured every five minutes via metabolism of methoxyresorufin to resorufin. Increases in resorufin concentration indicate activity. B) Averaged rates of production of MROD and BROD based on total cell number.

### DETAILED DESCRIPTION OF THE INVENTION

The development of implantable engineered liver tissue constructs and ex vivo hepatocyte based therapeutic devices are limited by an inadequate hepatocyte cell source. Differentiated pluripotent embryonic stem cells have been used to alleviate the cell source limitation problem but their utility is limited due to inefficiencies in generating the large number of cells required with sustained hepatocytoic function for extended periods of time. The present invention overcomes this by providing a hepatocyte lineage cell population developed from pluripotent stem cells that is able to maintain hepatocytic function during and post-incubation. Specifically, the present invention relates to isolated hepatocytic cells and cell populations and methods of producing them. In one embodiment, such cells are derived from differentiated stem cells and are matured using a morphogen, e.g. OSM or SNAP. In a further embodiment, the cells of the present invention are matured in a secondary culture (e.g. collagen sandwich culture) wherein the cells are incubated therein in the presence of the morphogen. In an even further embodiment, the cells of the present invention during and post-incubation exhibit characteristics of hepatocytic cells, namely intracellular ALB and CK18 expression and secretion as well as urea secretion. Additionally, the cells and populations of the present invention exhibit detoxification characteristics both during and post-incubation, wherein such characteristics were previously unseen in the starting stem cell population.

In a first embodiment, the hepatocytic cells of the present invention are derived from differentiated stem cells. Such differentiated stem cells may be derived from any multipotent, pluripotent, or totipotent stem cells known in the art. For example, the differentiated stem cells may be obtained from human embryonic stem cells, murine embryonic stem cells, or from other mammalian stem cells. Alternatively, stem cells may be obtained from human or murine umbilical cord blood or anyone other means associated with obtaining such cells. To this end, cells may be obtained from organisms, blastocysts, or cells isolated or created by suitable means known in the art. In other embodiments, the stem cells are adult stem cells, such as liver stem cells (e.g. oval cells), mesenchymal stem cells, pancreatic stem cells, multipotent adult stem cells and other stem cells that are able to give rise to hepatocyte-like cells when cultured according to a method described herein. Exemplary stem cells and methods of isolating such are described, e.g., in U.S. Pat. No. 5,861,313 by Pang et al. (pancreatic and hepatic progenitor cells); U.S. Pat. Nos. 6,146,889; 6,069,005; and 6,242,252 by Reid et al. (hepatic progenitor cells); and PCT International Patent Publication Nos. WO 01/11011 (multipotent adult stem cell lines); as well as WO 00/43498 and WO 00/36091 (human liver progenitor cells).

Any of the foregoing stem cell lines may be stored in a pluripotent, multipotent, totipotent, etc. state using media and methods known in the art for accomplishing such. For example, in one embodiment, the pluripotent stem cells may be stored in T-75 gelatin-coated flasks (Biocoat, BD-Biosciences, Bedford, Mass.) in Knockout Dulbecco's modified Eagles medium (Gibco, Grand Island, N.Y.) containing 15% knockout serum (Gibco), 4 mM L-glutamine (Gibco), 100 U/ml penicillin (Gibco), 100 U/ml streptomycin (Gibco), 10 ug/ml gentamicin (Gibco), 1000 U/ml ESGRO™ (Chemicon, Temecula, Calif.), 0.1 mM 2-mercaptoethanol (Sigma-Aldrich, St. Louis, Mo.). ESGRO™ contains leukemia inhibitory factor (LIF), which prevents embryonic stem cell differentiation.

Once differentiation is desired, the stem cells are exposed to primary culture conditions using methods known in the art for a sufficient amount to time to generate differentiated hepatocyte-like cells. Such conditions may be exposure of the stem cells to growth factors (e.g. FGF, EGF, HGF, HPO, nicotinamide, dexamethasone, insulin, etc.) in the presence of one or more known primary culturing medium. However, as previously provided, the use of such growth factors is entirely optional and not considered important to the present invention. While not limited thereto, in one embodiment, the stem cells are differentiated using techniques associated with Embryoid Body formation. Specifically, Embryoid bodies (EB) are formed by suspending the pluripotent cells in Iscove's modified Dulbecco's medium (Gibco) containing 20% fetal bovine serum (Gibco), 4 mM L-glutamine (Gibco), 100 U/ml penicillin, 100 U/ml streptomycin (Gibco), 10 ug/ml gentamicin (Gibco). The resulting Embryoid bodies are cultured for two days using the hanging drop method (1×10³ ES cells per 30 ul drop). The hanging drop is then cultured, such as by transferring the drop to suspension culture in 100 mm Petri dishes and culturing for an additional 2 days. The EB's are then plated, one EB per well, in 6 well tissue culture polystyrene plates (BD-Biosciences) for an additional 14-17 days. During this time, the EB cells spontaneously yield populations of hepatocyte lineage cells that, preferably, express one or more mature hepatocyte markers, e.g. albumin (ALB) and Cytokeratin 18 (CK-18).

As indicated above, the preferred incubation time of the EBs are 14-17 days, although the present invention is not limited thereto. While the EB cells are preferably selected for secondary culture at any point after 14 days, cells from day 17 EB's are most preferable because they have been observed to have the greatest hepatocyte function at that time.

The method of achieving a differentiated stem cell, however, is not limiting to the foregoing. Rather, one of ordinary skill in the art will understand that any method of differentiating cells may be used so as to arrive at the differentiated stem cells of the present invention. For example, in one embodiment such differentiation may be obtained using a monolayer platform such as that described in Sharma et al. 2006, the contents of which are incorporated by reference herein. In another embodiment, the stem cell differentiation into hepatocyte-like cells may be obtained using an encapsulation platform such as that described in Maguire et al. 2006, the contents of which are incorporated herein by reference. In an even further alternative, the differentiated cells of the present invention may be created using any other EB mediated platform such as those of Hamazaki et al. 2001; Heo et al. 2006; Kumashiro et al. 2005b, the contents of which are incorporated herein by reference. To this end, the method of preparing the intermediate differentiated cells of the present invention are not intended to limit the present invention and one of ordinary skill in the art may use any method or system of deriving the same.

Once the differentiated stem cells are obtained, using any of the above methodologies, these cells are then matured into a hepatocytic cell line that maintains hepatocytic activity during and post-incubation. More specifically, in one embodiment the differentiated cells are plated onto a secondary culture. In a preferred embodiment, the secondary culture is comprised of a collagen-based matrix. For example, in one embodiment the secondary culture is a collagen sandwich culture.

In an even further embodiment, the differentiated stem cells are incubated in the secondary culture in the presence of a morphogen. The term “morphogen,” as used herein, refers to a compound that facilitates and/or directs tissue differentiation. In the present invention, the morphogens contemplated include S-NitrosoAcetylPenicillamine (SNAP) and Oncostatin-M (OSM). To this end, differentiated stem cells are matured in the presence of either SNAP or OSM. In a most preferred embodiment, the differentiated EB cells discussed above are matured in a collagen sandwich culture in the presence of either S-NitrosoAcetylPenicillamine (SNAP) and Oncostatin-M (OSM).

As used herein, S-NitrosoAcetylPenicillamine (SNAP) refers to a chemical compound with the chemical formula ONSC(CH₃)₂CH(NHAc)CO₂H wherein O refers to an oxygen atom, N refers to a nitrogen atom, S refers to a sulfur atom, C refers to a carbon atom, H refers to a hydrogen atom, and Ac refers to an acetyl group having the formula COCH₃. As also used herein, Oncostatin-M refers to a pleiotropic cytokine belonging to the Interleukin 6 group of cytokines.

In accordance with the foregoing, in one embodiment of obtaining the hepatocytic cell of the present invention, the previously differentiated stem cells, e.g. EB cells, are isolated from their primary differentiation culture, discussed above, and are re-plated into a collagen-matrix coated plate, preferably a multi or six well plate. In one embodiment, the total volume of collagen-matrix in each well is approximately 2 ml. Isolation of the previously differentiated EB cells may be performed using any standard method known in the art. In one embodiment, such isolation entails incubating the cells within 0.5 ml of trypsin (Gibco) for three minutes, resulting in a single cell suspension, and subsequently adding IMDM media. However, the present invention is not limited to this particular technique and any other known technique may be employed.

In a further embodiment, the six well plate is preferably a 6 well tissue culture polystyrene (BD-Biosciences) wherein the collagen coating the wells is comprised of rat tail type I collagen (BD-Biosciences) gels, or any similar collagen gels, prepared by distributing 350 μL of collagen gel solution (3 parts 1.33×DMEM, pH 7.4, and 1 part collagen solution at 4 mg/mL, chilled on ice and mixed immediately prior to use) evenly over one well of a six well plate (BD-Biosciences) and incubated at 37° C. for at least one hour before use. This plate, collagen structure and composition, however, are not limiting to the present invention and one of ordinary skill will appreciate the interchangeability of other such plates and collagen matrix in accordance with the objectives of the present invention.

Regardless of the equipment use, in one embodiment, once isolated, the differentiated stem cells, preferably EB cells, are thereafter plated onto the collagen matrix at an initial seeding density corresponding to a density of 5×10⁴ day 17 cells per well of a six-well plate. Generally, it may be desirable to let the cells proliferate in order to generate the large mass. However, if the cell proliferation rate is not fast enough, it may be beneficial to increase the initial seeding density to facilitate cell-cell contact. For example, in a six-well plate, the surface area can hold up to ˜5.0×10⁶ at 95% confluence. Based on the fact that the inventors have plated about 5×10⁴ cells on day 1 and obtained ˜1.0×10⁶ by day 10, as discussed in the Examples, the initial seeding density can be increased up to five fold range and the range is from about 5×10⁴ to about 2.5×10⁵. The about 2.5×10⁵ cell number should theoretically produce 95% confluence by day 10 and may increase hepatocytic function due to the increase in cell-cell contact. Once re-plated, the cells are allowed to seed to the collagen for approximately 24 hrs at 37° C., whereafter the media is aspirated and a second layer of the foregoing collagen is added as a top layer. This, in effect, creates a “collagen sandwich” with the differentiated stem cells in the middle.

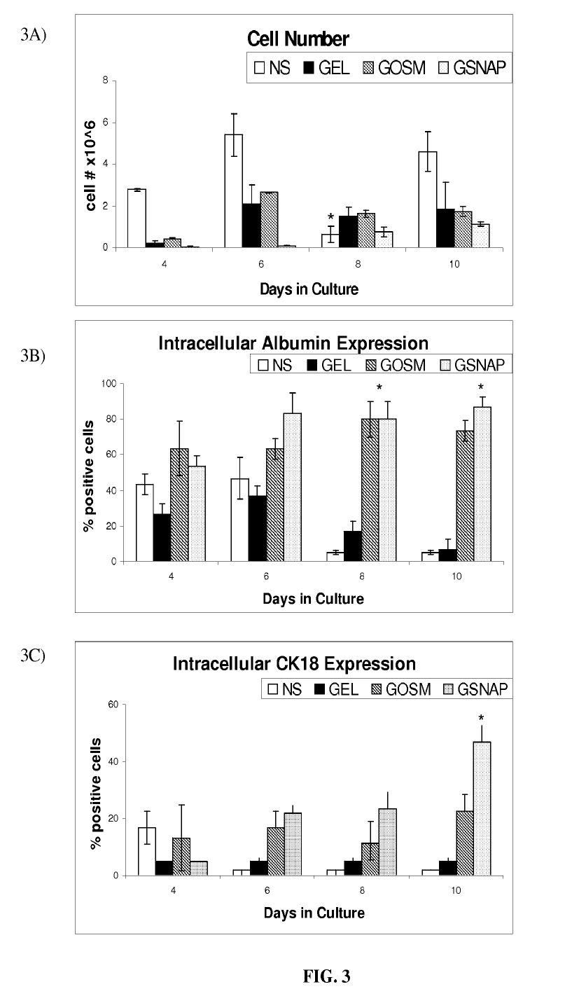

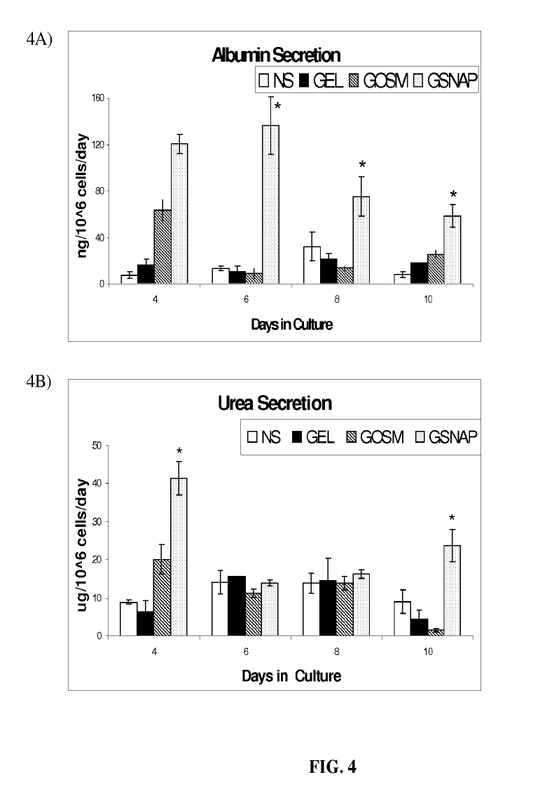

While not limited thereto, in one embodiment, the morphogen is added after the formation of the collagen sandwich configuration. In a further embodiment, approximately 250 μM SNAP is added to the collagen sandwich. The acceptable dose range of SNAP is 50-500 μM SNAP for varied tissue-specific embryonic stem cell differentiation beyond which a significant loss in cell viability is observed. In an alternative embodiment, approximately 10 ng/ml OSM is added to the collagen sandwich. OSM is typically used at a concentration of 10 ng/ml for fetal hepatocyte and committed embryonic stem cell derived hepatic cell maturation. A suitable range (i.e., the dose response relationship) of OSM concentrations can be obtained with no more than a routine experimentation within the expertise of a skilled artisan. In an even further embodiment any amount of SNAP and/or OSM may be added to the collagen sandwich configuration so as to effectuate hepatocyte cell line maturation in accordance with the present invention herein. The differentiated EB cells are then incubated for a time period sufficient to produce mature hepatocyte cell lines that exhibit and maintain heptocytic function (e.g. Albumin expression/secretion, Cytokeratin expression/secretion, and/or urea secretion) and detoxification. In one embodiment, such incubation period is at least 6 days, with a preferred range between 6-10 days incubation. In a most preferred embodiment, and as further illustrated in FIGS. 3 and 4 using the foregoing methods and measurements, between 10%-60% of the cell population exhibit and maintain heptocytic function and detoxification after six (6) days incubation.

Post-incubation, the final cells may be removed from the secondary culture using any known methods in the art. In one embodiment, the cells are isolated by first being washed in PBS (Gibco) and then dissociated from the collagen with 0.5 mL of 0.1% collagenase (Sigma-Aldrich) in PBS for minutes at 37° C. before being re-plated onto other suitable surfaces depending on the choice of the user of the methods of the instant invention. For example, for determination of the presence and/or extent of hepatocytic functions, the cells may be re-plated into 12 well plates. The present invention, using at least the foregoing methods and applications, is advantageous because the hepatocytic cell lines and cell populations produced maintain hepatocytic activity both during and after the secondary incubation phase. Exemplary populations of cells comprised at least 10-60% of cells generated having hepatocytic activity. Such hepatocytic activity includes, but is not limited to, albumin expression and secretion, Cytokeratin 18 expression and secretion, urea secretion, and detoxification (particularly by way of CYP450 metabolism). To this end, hepatocyte activity of the cells of the present invention can be characterized in that the cells are (1) positive for late stage markers of hepatocytes, e.g. HNF-1α, cytokeratin (CK) 18 and albumin; (2) negative for early hepatocyte markers, e.g., HNF-3β, GATA4, CK19, α-fetoprotein; express cytochrome P450 genes, e.g., CYP1A1, CYP2B1, CYP2C6, CYP2C11, CYP2C13, CYP3A2 and CYP4A1; and acquire a polarized structure. Other markers used for detection of hepatocyte cells of the present invention include α1-antitrypsin, glucose-6-phosphatase, transferrin, asialoglycoprotein receptor (ASGR), CK7, γ-glutamyl transferase; HNF 1β, HNF 3α, HNF-4α, transthyretin, CFTR, apoE, glucokinase, insulin growth factors (IGF) 1 and 2, IGF-1 receptor, insulin receptor, leptin, apoAII, apoB, apoCIII, apoCII, aldolase B, phenylalanine hydroxylase, L-type fatty acid binding protein, transferrin, retinol binding protein, and erythropoietin (EPO).

In an even further alternative, hepatocyte-like cells may also display the following biological activities, as evidenced by functional assays. The cells may have a positive response to dibenzylfluorescein (DBF); have the ability to metabolize certain drugs, e.g., dextromethorphan and coumarin; have drug efflux pump activities (e.g., P glycoprotein activity); upregulation of CYP activity by phenobarbital, as measured, e.g., with the pentoxyresorufin (PROD) assay, which is seen only in hepatocytes and not in other cells (see, e.g., Schwartz et al., J. Clin. Invest., 109:1291 (2002)); take up LDL, e.g., Dil-acil-LDL (see, e.g., Schwartz et al., supra); store glycogen, as determined, e.g., by using a periodic acid-Schiff (PAS) staining of the cells (see, e.g., Schwartz et al., supra); produce urea and albumin (see, e.g., Schwartz et al., supra); and present evidence of glucose-6-phosphatase activity.

Previously, there have been few successful reports of maintaining EB derived hepatocyte like function after the primary differentiation is complete. In fact, most studies do not explore function past the initial differentiation protocol. As noted above, a significant problem associated with ex vivo adult hepatocyte culture is the rapid loss of differentiated function and morphology. (Koide et al. 1989; Nahmias et al. 2007). The present invention, thus, provides that such loss may be cured through the use of a morphogen, e.g. SNAP or OSM, when cultured in collagen sandwich configuration. As provided in the examples below, the combination of sandwich culture with SNAP or OSM supplementation provided differentiated cells that maintained hepatocyte function for weeks post-incubation. For example, such incubation resulted in expression at all time points with over 80% ALB positive cells indicating a relatively homogeneous population about four weeks after differentiation induction. In certain embodiments, at least 10% of the cells of the present invention exhibited positive hepatocyte-like activity (e.g. CK 18 expression) after six days, eight days, or ten days in the supplemented secondary culture. In further embodiments, the cells of the present invention are characterized by secretion of albumin in an amount over 40 ng per 10⁶ cells per day after about 10 days in the supplemented secondary culture. In other embodiments, the cells of the present invention are characterized by secretion of urea in an amount of at least 15 ng per 10⁶ cells per day after about 10 days in the supplemented secondary culture. In a further embodiment, the cells of the present invention, after being cultured for at least about 10 days in supplemented secondary media, are characterized by having cytochrome P450 activity corresponding to at least about 200 uM/ml resorufin after 30 minutes. These functions are by no means limiting to the examples, but are set forth to illustrate the hepatocytic characteristics of the cells and cells populations of the present invention.

In addition to maintaining ALB, CK18 and urea secretion, the forgoing methods and resulting cells induced albumin secretion not seen at any significant level in day EB culture. While Albumin secretion from ES derived hepatocyte lineage cells has been reported previously (Gouon-Evans et al. 2006; Maguire et al. 2007; Soto-Gutierrez et al. 2006; Teratani et al. 2005a; Tsutsui et al. 2006), in the current studies it is first detected at day four at 120 ηg/10⁶ cells/day and decreases to about 60 ηg/10⁶ cells/day. Although this is significantly lower than the levels of secretion seen in the Hepa1-6 control, it is significantly higher than any other experimental condition evaluated here and similar to previously reported ES derived hepatocyte-like secretion level.

The present cells are also advantageous because they provide for detoxification characteristics. Specifically, in one embodiment, CYP450 metabolism is detectable in differentiated cells cultured with SNAP. While xenobiotic metabolism has been well characterized in primary hepatocyte systems, (Behnia et al. 2000; Roy et al. 2001) and although there have been reports of induction of CYP450 mRNA in ES derived hepatocyte-like cells, there have been few reports detoxification, a function which would be critical for use of these cells in a LAD. (Asahina et al. 2004; Soto-Gutierrez et al. 2006; Tsutsui et al. 2006)

The present invention, including the foregoing methods, systems and advantages has a plurality of uses. For example, in one embodiment, hepatocyte-like cells may be used in drug studies to test the effects and or metabolic breakdown of a prior or potential drug by hepatocytic cell or to screen the effect of certain compounds on the cell types. Alternatively, the cells of the present invention may be used as a biological tool testing the biological effects of a particular compound on the drug. Such studies may be adapted based upon known protocols for the particular class of drugs or known experimental techniques for testing such effects. To this end, the cells of the present invention may easily be adapted to screening assays for similar purposes.

As an alternative to drug and biological screening methods, the hepatocyte-like cells of the present invention may also be administered to a subject in need thereof. Specifically, cells of the present invention may be cultured ex vivo, then administered to the liver of the subject for tissue reconstitution or regeneration. The tissue construct may be administered to a patient suffering from mild to severe liver damage or acute liver failure. Such constructs may be formulated in any conventional manner using one or more physiologically acceptable carriers comprising excipients and auxiliaries which facilitate processing of the compounds into preparations which can be used pharmaceutically. Proper formulation is dependent upon the route of administration chosen. Hepatocyte-like cells can be used in therapy by direct administration, or as part of a bioassist device that provides temporary liver function while the subject's liver tissue regenerates itself following fulminant hepatic failure. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The compositions may be packaged with written instructions for use of the cells in tissue regeneration, or restoring a therapeutically important metabolic function.

In furtherance of the foregoing tissue constructs including cells of the present invention may further include bioartificial liver support (BAL) such as extracorporeal liver assist devices (LAD). In accordance with the foregoing, tissue constructs, BALs, LADs, and other similar medical devices may be used in conjunction with the cell populations of the present invention in any conventional manner known by one of ordinary skill in the art.

### EXAMPLES

The present invention is further illustrated by the following examples, which should not be construed as limiting in any way.

### Example 1

### Cell Cultures

All cell cultures were incubated in a humidified 37° C., 5% CO₂ environment. The ES cell line D3 (ATCC, Manassas, Va.) was maintained in an undifferentiated state in T-75 gelatin-coated flasks (Biocoat, BD-Biosciences, Bedford, Mass.) in Knockout Dulbecco's modified Eagles medium (Gibco, Grand Island, N.Y.) containing 15% knockout serum (Gibco), 4 mM L-glutamine (Gibco), 100 U/ml penicillin (Gibco), 100 U/ml streptomycin (Gibco), 10 ug/ml gentamicin (Gibco), 1000 U/ml ESGRO™ (Chemicon, Temecula, Calif.), 0.1 mM 2-mercaptoethanol (Sigma-Aldrich, St. Louis, Mo.). ESGRO™ contains leukemia inhibitory factor (LIF), which prevents embryonic stem cell differentiation. Every 2 days, media was aspirated and replaced with fresh media. Cultures were split and passaged every 6 days, following media aspiration and washing with 6 ml of phosphate buffered solution (PBS) (Gibco). Cells were detached following incubation with 3 ml of trypsin (Gibco) for three minutes, resulting in a single cell suspension, and subsequently the addition of 12 ml of Knockout DMEM. Cells were then replated in gelatin-coated T-75 flasks at a density of 1×10⁶ cells/ml. Staining with Oct4, a recognized stem cell marker, demonstrated that the cells remain undifferentiated over the period used to accomplish these studies. 100% Oct4 staining was observed at all passages.

In order to induce differentiation, cells were suspended in Iscove's modified Dulbecco's medium (Gibco) containing 20% fetal bovine serum (Gibco), 4 mM L-glutamine (Gibco), 100 U/ml penicillin, 100 U/ml streptomycin (Gibco), 10 ug/ml gentamicin (Gibco). Embryoid bodies were formed and cultured for two days using the hanging drop method (1×10³ ES cells per 30 ul drop). Hanging drops where transferred to suspension culture in 100 mm petri dishes and cultured for an additional 2 days. The EB's were then plated, one EB per well, in 6 well tissue culture polystyrene plates (BD-Biosciences) for an additional 14 days. For secondary culture day 17 EBs cells were detached following incubation with 0.5 ml of trypsin (Gibco) for three minutes, resulting in a single cell suspension, and subsequently the addition of IMDM media. Cells from day 17 EB's were used because it has been observed that hepatocyte function is greatest on day 17. Cells were then re-plated in 6 well tissue culture polystyrene (BD-Biosciences) at an initial seeding density of 5×10⁴ day 17 cells per well for further analysis. Culture medium was changed every forty eight hours. When OSM and SNAP were supplemented, 10 ng/ml OSM and 250 μM SNAP were added to the culture medium. When collagen sandwich culture was used, rat tail type I collagen (BD-Biosciences) gels were prepared by distributing 350 μL of collagen gel solution (3 parts 1.33×DMEM, pH 7.4, and 1 part collagen solution at 4 mg/mL, chilled on ice and mixed immediately prior to use) evenly over one well of a six well plate (BD-Biosciences) and incubated at 37° C. for at least one hour before use. 5×10⁵ cells were seeded in 2 mL of IMDM media on day 0 and an additional 350 μL of collagen gel solution was distributed over the cells after 1 day of culture. Therefore, the second layer of collagen is added on day 1 of secondary culture protocol. One hour of incubation at 37° C. was allowed for gelation and attachment of the second gel layer before the medium was replaced. Culture medium was changed every forty eight hours.

The Hepa 1-6 cell line (ATCC, Manassas, Va.) was maintained in Dulbecco's modified Eagles medium (Gibco) containing 10% fetal bovine serum (Gibco), 100 U/mL penicillin (Gibco), 100 U/mL streptomycin (Gibco), and 4 mM L-glutamine (Gibco). Hepa 1-6 cells were grown on tissue culture treated T-75 flasks (Falcon, BD Biosciences, San Jose, Calif.). Hepa 1-6 cells were used as positive controls for each of the following assays.

On evaluation days 4, 6, 8 and 10 days in secondary culture, cells were re-plated into 12 well plates. Media samples were collected after 24 hours of culture at 37° C. and 5% CO₂. The cells were then washed in PBS (Gibco) and fixed in 4% paraformaldehyde (Sigma-Aldrich) in PBS for 15 min at room temperature. Cells in collagen sandwich culture were dissociated with 0.5 mL of 0.1% collagenase (Sigma-Aldrich) in PBS for 30 minutes at 37° C. before re-plating into 12 well plates.

### Example 2

### In Situ Indirect Immunofluorescent Cytokeratin-18 and Intracellular Albumin Analysis

After 24 hours in culture and fixing with 4% paraformaldehyde, the cells were then washed for 10 min in cold PBS and fixed in 4% paraformaldehyde (Sigma-Aldrich) in PBS for 15 minutes at room temperature. The cells were washed twice for 10 min in cold PBS and then twice for 10 min in cold saponine/PBS (SAP) membrane permeabilization buffer containing 1% bovine serum albumin (BSA) (Sigma-Aldrich), 0.5% saponine (Sigma-Aldrich) and 0.1% sodium azide (Sigma-Aldrich). To detect intracellular albumin, the cells were subsequently incubated for 30 minutes at 4° C. in a SAP solution containing rabbit anti-mouse albumin antibody (150 ug/ml) (MP Biomedicals, Irvine, Calif.), or normal rabbit serum (150 ug/ml) (MP Biomedicals) as an isotype control, washed twice for 10 min in cold SAP buffer, and then treated for 30 minutes at 4° C. with the secondary antibody, FITC-conjugated donkey anti-rabbit, diluted 1:500 (Jackson Immuno Labs, Westgrove, Pa.). To detect cytokeratin 18, which is produced in mature hepatocytes and a few other mature cell types, cells we incubated for 30 minutes at 4° C. in a SAP solution containing rabbit anti-moue cytokeratin 18 antibody (IgG1) (1:50 dilution) (Santa Cruz Biotechnology) or the IgG1 fraction of normal rabbit serum (1:100 dilution) (Santa Cruz Biotechnology) as an isotype control, and then treated for 30 minutes at 4° C. with the secondary antibody, FITC-conjugated goat anti-rabbit, diluted 1:200 (Jackson Immuno Labs, Westgrove, Pa.). For both stains, cells were then washed once with cold SAP buffer and once with cold PBS. Fluorescent images were acquired using a computer-interfaced inverted Olympus IX70 microscope. Specimens were excited using a 515 nm filter. Fluorescent intensity values were determined for each cell using Olympus Microsuite. Experimental intensity values for each cell were calculated after subtracting the average intensity of the isotype control.

### Example 3

### Sandwich ELISA for Detection of Albumin Secretion

In order to detect secreted albumin within the media supernatants obtained on each of the analysis days, we used a commercially available mouse albumin ELISA kit (Bethyl Laboratories, #E90-134). A standard curve was generated by creating serial dilutions of an albumin standard from 7.8 to 10,000 ng/mL. Absorbance readings were obtained using a Biorad (Hercules, Calif.) Model 680 plate reader with a 450 nm emission filter. Albumin values were normalized to the cell number recorded on the day of media sample collection.

### Example 4

### Urea Secretion

Media samples were collected on all analysis days. Urea synthesis was assayed using a commercially available kit (StanBio, Boerne, Tex.). A standard curve was generated by creating serial dilutions of a urea standard from 0 to 300 mg/mL. Absorbance readings were obtained using a Biorad (Hercules, Calif.) Model 680 plate reader with a 585 nm emission filter. Urea values were normalized to the cell number recorded on the day of media sample collection.

### Example 5

### Measurement of Cytochrome P450 Activity

On evaluation days 4, 6, 8 and 10 days in secondary culture, cells were re-plated into 12 well plates. 3-Methylcholanthrene was used at a concentration of 2 μM (Sigma-Aldrich) for 48 hours prior to the addition of resorufin as an inducer of cytochrome P450 activities. Cytochrome P450-dependent resorufin o-dealkylase activity (BROD, PROD, EROD, and MROD) was measured using resorufin substrates namely pentoxy-, benzyloxy-, ethoxy-, and methoxyresorufin from a Resorufin Sampler Kit (Invitorgen, Carlsbad, Calif.). The incubation mixture contained resorufin substrates (pentoxy-, ethoxy-, or methoxyresorufin, final concentration 5 mM) and dicumarol (80 mM) in phenol red free Earle's Balanced salt Solution (EBSS) (Gibco). The prepared solutions were preheated to 37° C., prior to incubation with cells. The 12 well plates were washed with 2 mL of EBSS (37° C.) and further incubated with 2 mL of EBSS at 37° C. for 5-7 min, to remove the residual medium. Following removal of EBSS, the incubation mixture was added (2 mL per well), and the dishes were incubated at 37° C. in a 5% CO2 incubator. At various time points (5, 10, 15, 20, 25 min) following incubation, 100 μL of the mixture was transferred into a 96-well plate. The fluorescence of the plate was measured using a fluorescence plate reader (DTX880, Beckman Coltour, Fullerton, Calif., ext. 530 nm and emis. 590 nm) at the end of min incubation. A standard curve of resorufin fluorescence was constructed using concentrations ranging from 1 to 1,000 nmol in EBSS. A linear curve was obtained with an r² of 0.99. The constructed standard curve was used to convert the fluorescence values obtained from the plate reader to nanomoles of resorufin. Rate of formation of resorufin, as calculated from the early linear increase in the fluorescence curve, was defined as cytochrome P450 activity and expressed as nmol/min.

### Example 6

### Statistical Analysis of Functional Assays

Each data point represents the mean of three experiments (each with three biological replicates), and the error bars represent the standard deviation of the mean. Statistical significance was determined using the student t-test for unpaired data. Differences were considered significant when the probability was less then or equal to 0.05.

### Example 7

### Dynamic Studies of Secondarily Cultured EB Derived Hepatocyte Lineage Cells

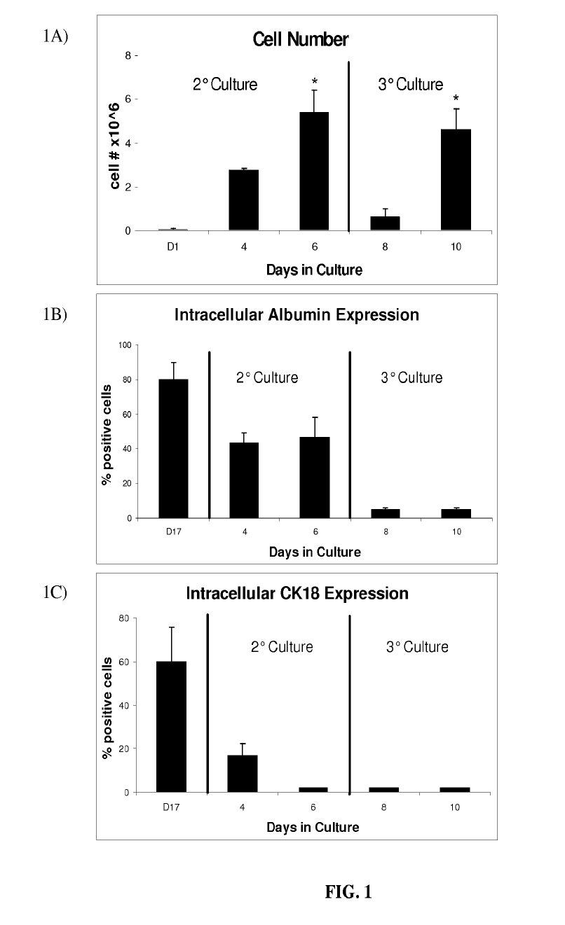

Previous studies demonstrated that EB mediated differentiation of ES cells spontaneously yield a population of cells displaying specific hepatocyte characteristics such as albumin and CK-18. However, secondary culturing of these cells in standard tissue culture conditions resulted in the loss of these specific hepatocyte functions. Therefore, cultures were established to study the maintenance and augmentation of hepatocyte like function previously observed after 17 days of spontaneous EB mediated differentiation. Hepatocyte lineage maintenance was initially assessed by examining the dynamics of cell growth following removal of cells from their primary EB culture and re-plating into tissue culture polystyrene. 5×10⁴ cells from day 17 EB cultures were re-plated into one well of a six well plate and evaluated on days 4, 6, 8 and 10 days post re-plating. Cell number increased rapidly and confluence was reached at day 6. Therefore, cells were re-plated into tertiary culture at 5×10⁴ cells per well and continued to proliferate for the next four days. (FIG. 1A).

Next, experiments were designed to evaluate the maintenance of function seen in EB generated hepatocyte lineage cells by assessing in situ intracellular ALB and CK18 expression. Secondary and tertiary cultures were initiated as outlined above and ALB and CK18 expression were qualitatively assessed 4, 6, 8 and 10 days post re-plating using indirect immunofluorescence with either primary anti-ALB/CK18 antibody or an immunoglobulin control serum and subsequently fluorescently labeled secondary antibody. Images were captured using digital microscopy in order to determine the percent of ALB and CK18 expressing cells within the cultures. Day 17 EB generated cells were 80% albumin positive (FIG. 1B) and 60% CK18 positive (FIG. 1C). As depicted in FIG. 1B, ALB expression was maintained for 6 days at ˜40% in secondary culture, however, expression was not maintained past 6 days in tertiary culture. CK18 expression was maintained at minimal expression levels for 4 days in secondary culture but was absent on subsequent days. (FIG. 1C) In secondary polystyrene culture, EB derived cells proliferated rapidly but could not sustain albumin expression. The addition of soluble factors on proliferation and maintenance of function was explored.

### Example 8

### OSM and SNAP Supplementation

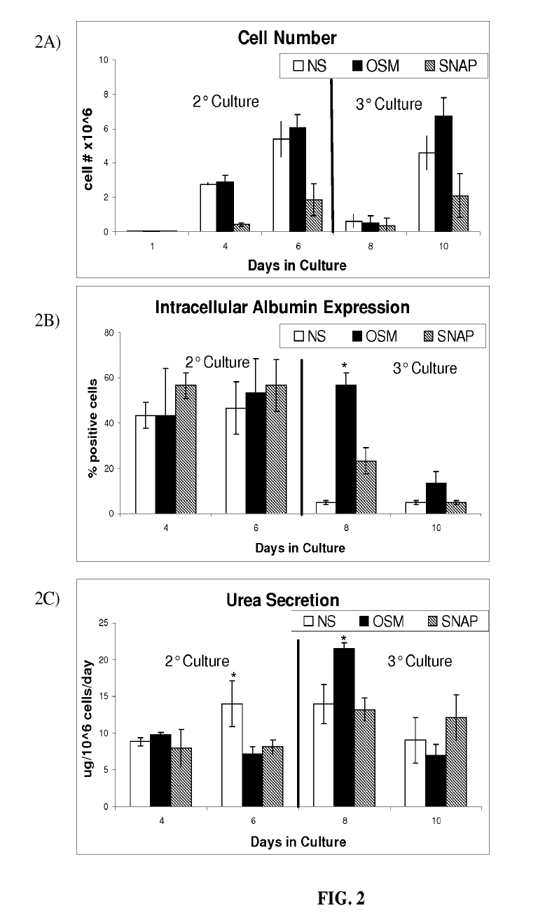

In order to investigate the effect of soluble factors previously shown to affect hepatic function, re-plated cells were supplemented with either OSM or SNAP. Cell numbers in the OSM supplemented condition were similar to that of the un-supplemented cultures and increased dramatically in the OSM supplemented cultures. However, cells exposed to SNAP were generally characterized by slower growth rates. Due to the rapid growth seen in the un-supplemented and OSM cultures, at day 6, cells were re-plated into tertiary culture at 5×10⁴ cells per well and continued to proliferate for the next four days. (FIG. 2A) ALB expression was maintained in the OSM supplemented cultures for up to eight days in secondary culture. The cells supplemented with SNAP also maintained some ALB expression up to eight days in tertiary culture but at a lower level. There was no significant expression following ten days in secondary culture in any condition. (FIG. 2B) Urea secretion was greatest at day eight in the OSM supplemented condition however some secretion was detected at all experimental time points. Day 17 EB hepatocyte like cells exhibited a urea secretion rate of 50 μg/10⁶ cells/day. (FIG. 2C) A summary of the hepatocyte like functions tested is summarized in Table 1, below. CK18 expression as well as other hepatocyte functions such as albumin secretion, glycogen storage and CYP450 mediated detoxification was not detected at any level in the OSM or SNAP supplemented cultures. Although addition of soluble factors maintained albumin secretion for up to eight days in secondary culture, certain hepatocyte functions were not maintained at any significant level and others were totally absent.

TABLE 1

| | NS | SNAP | OSM |
|---|---|---|---|
| Intracellular ALB | + | + | + |
| CK18 | + | − | − |
| Urea | + | + | + |
| ALB Secretion | − | − | − |
| CYP450 | − | − | − |
| Glycogen | − | − | − |

A (+) represents at least one time point of significant expression or secretion function in the three conditions.
A (−) represents functions which were absent on all experimental days.

### Example 9

### Collagen Sandwich Culture

In order to determine whether it was possible to further augment and/or maintain the function of the hepatocyte like cells isolated from day 17 EB culture, collagen sandwich culture, a system which has been well studied for maintenance of mature hepatocyte function, (Dunn et al. 1989) was utilized alone (GEL) and in conjunction with OSM (GOSM) and SNAP (GSNAP) supplementation. Cells cultured in a sandwich configuration were characterized by a slower rate of proliferation as compared to polystyrene culture. While the proliferation rate of SNAP treated cells is significantly lower, cellular function better resembles that of adult hepatocytes, which do not proliferate in vitro. Cells in the GEL and GOSM conditions reached maximum growth at day 6 and cells in GSNAP by day 8. Because of low proliferation rates in sandwich culture the cells did not reach absolute confluence and no tertiary culture was employed. (FIG. 3A) As depicted in FIG. 3B, albumin expression was detected in all conditions for 6 days in secondary culture, however, expression was maintained at 80%, 10 days post re-plating, only in the GSNAP and GOSM conditions. Although, the combination of collagen gel cultures and SNAP on day 10 of secondary cell culture yields an 8 fold increase in albumin positive cell population (FIG. 3B) over the control, the cell number is 5 five fold lower (FIG. 3A). A similar effect was evident in the expression of CK18 where some expression was detected in the non supplemented, non GEL (NS) condition for 4 days but it was expressed only in the GSNAP and GOSM conditions after 10 days. However, the ˜45% expression in the GSNAP condition was significantly higher than the ˜20% seen in the GOSM condition. (FIG. 3C) The GEL, GOSM and GSNAP conditions stored glycogen 10 days into secondary culture. The NS conditions did not significantly stain for glycogen.

Urea and albumin secretion, vital liver functions, were used to asses mature hepatocyte specific differentiated function. A dynamic profile of ALB secretion was established using qualitative ELISA analysis. Although at four days in secondary culture there was an initial induction of ALB secretion in both the GOSM and GSNAP conditions, rates were significantly higher in the GSNAP condition on subsequent days compared to all other conditions. In addition, secretion was maintained at 60 ηg/10⁶ cells/day after 10 days in secondary culture (FIG. 4A). Day 17 EB derived cells did not secrete albumin (data not shown) and had a urea secretion rate of 50 μg/10⁶ cells/day. In secondary culture all conditions maintained some urea secretion. However, the 25 μg/10⁶ cells/day observed in the GSNAP condition was significantly higher than any other condition at day 10 (FIG. 4B).

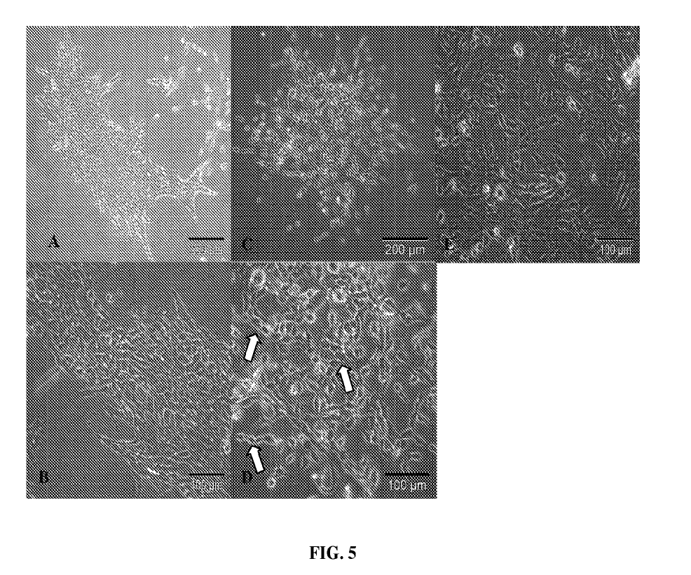

At the end of the culture period, cells cultured in all conditions were characterized by a variety of cell morphologies. Cells were assembled in random densely packed groupings in all double gel conditions and exhibited tightly packed morphologies. However, in the GSNAP condition, there was a second morphology which was characterized by greater than 95% of cells in groups of round or square cells in a non confluent, loosely connected environment (FIG. 5).

### Example 10

### Cytochrome P450 Detoxification

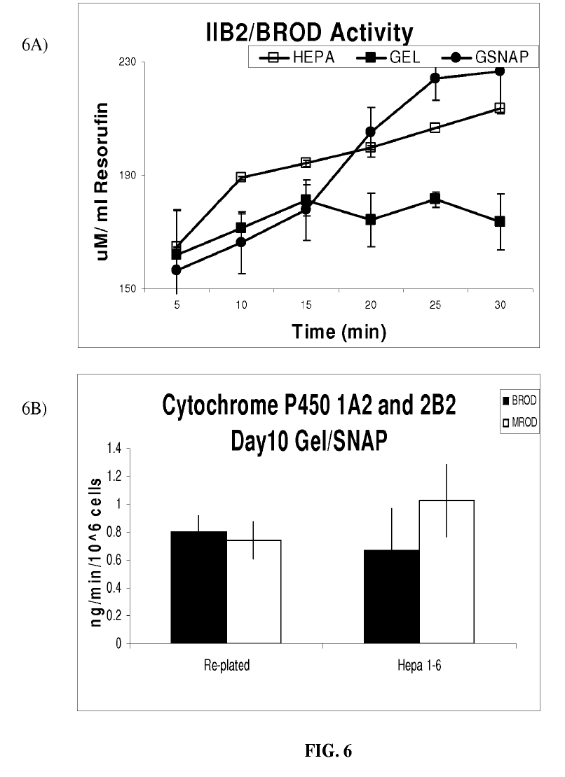

Cytochrome P450 enzymes play a key role in detoxifying xenobiotics and were used in these studies to asses hepatocyte function. The present studies monitored the expression and stabilization of benzyloxyresorufin o-dealkylase (BROD) and methoxyresorufin o-dealkylase following induction with 3-methylcholanthrene for 48 hrs, in D17 EB derived cells as and for 10 days in secondary GSNAP culture. BROD and MROD activity can be determined from the enzymatic conversion of resorufin. This activity detected via increasing concentration of resorufin was only apparent after 10 days in secondary GSNAP culture. (FIG. 6A) The rate of production was similar to that of the Hepa 1-6 control (FIG. 6B).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated herein by reference.

1. An isolated cell comprising
a maturated hepatocytic stem cell exhibiting detoxification characteristics wherein the hepatocytic stem cell is maturated from a differentiated stem cell cultured in the presence of S-NitrosoAcetylPenicillamine or Oncostatin-M.

2. The isolated cell of claim 1 wherein the differentiated stem cell is selected from the group consisting of human embryonic stem cells, murine embryonic stem cells, and human umbilical cord cells.

3. The isolated cell of claim 1 wherein the differentiated stem cell is an embryoid body-mediated heptaocyte-like stem cell.

4. The isolated cell of claim 1 wherein the maturated hepatocytic stem cell exhibits CYP expression so as to facilitate detoxification.

5. The isolated cell of claim 1 wherein the differentiated stem cell is cultured in or on a collagen matrix.

6. The isolated cell of claim 1 wherein the differentiated stem cell is cultured in a collagen sandwich configuration.

7. A method of producing a maturated hepatocytic cell comprising providing a differentiated stem cell having heptocyte-like characteristics; and culturing the differentiated stem cell in the presence of S-NitrosoAcetylPenicillamine or Oncostatin-M.

8. The method of claim 7 wherein the differentiated stem cell is selected from the group consisting of human embryonic stem cells, murine embryonic stem cells, and human umbilical cord cells.

9. The method of claim 7 wherein the differentiated stem cell is an embryoid body-mediated heptocyte-like stem cell.

10. The method of claim 7 wherein the maturated hepatocytic stem cell exhibits CYP expression so as to facilitate detoxification.

11. The method of claim 7 wherein the differentiated stem cell is cultured in or on a collagen matrix.

12. The method of claim 7 wherein the differentiated stem cell is cultured in a collagen sandwich configuration.

14. An implantable tissue construct comprising maturated hepatocytic stem cells exhibiting hepatocyte-like characteristics and detoxification characteristics wherein the hepatocytic stem cells are maturated from a differentiated stem cell cultured in the presence of S-NitrosoAcetylPenicillamine or Oncostatin-M.

15. A tissue construct for use in extracorporeal liver assist devices comprising; maturated hepatocytic stem cells exhibiting hepatocyte-like characteristics and detoxification characteristics wherein the hepatocytic stem cells are maturated from a differentiated stem cell cultured in the presence of S-NitrosoAcetylPenicillamine or Oncostatin-M.

16. A population of cultured cells, derived from embryoid bodies, wherein within said population:
a) at least about 10% of cells comprising said population are positive for intracellular CK 18 expression after six days in culture; or
b) at least about 10% of cells comprising said population are positive for intracellular CK 18 expression after eight days in culture; or
c) at least about 10% of cells comprising said population are positive for intracellular CK 18 expression after ten days in culture.

17. The population of claim 16, wherein
a) at least about 15% of cells comprising said population are positive for intracellular CK 18 expression after six days in culture; or
b) at least about 15% of cells comprising said population are positive for intracellular CK 18 expression after eight days in culture; or
c) at least about 15% of cells comprising said population are positive for intracellular CK 18 expression after ten days in culture.

18. The population of claim 17, wherein
a) at least about 20% of cells comprising said population are positive for intracellular CK 18 expression after six days in culture; or
b) at least about 20% of cells comprising said population are positive for intracellular CK 18 expression after eight days in culture; or
c) at least about 20% of cells comprising said population are positive for intracellular CK 18 expression after ten days in culture.

19. The population of claim 16, wherein no more than about 30% of cells comprising said population are positive for intracellular CK 18 expression.

20. The population of claim 18, wherein at least about 30% of cells comprising said population are positive for intracellular CK 18 expression after ten days in culture.

21. The population of claim 20, wherein at least about 40% of cells comprising said population are positive for intracellular CK 18 expression after ten days in culture.

22. The population of claim 20, wherein no more than about 60% of cells comprising said population are positive for intracellular CK 18 expression.

23. A population of cultured cells, derived from embryoid bodies, said population characterized by secretion of albumin in an amount of at least about 40 ng per 10⁶ cells per day after about ten days in culture.

24. The population of claim 23, characterized by secretion of albumin in an amount of no more than about 70 ng per 10⁶ cells per day after about ten days in culture.

25. A population of cultured cells, derived from embryoid bodies, said population characterized by secretion of urea in an amount of at least about 15 ng per 10⁶ cells per day after about ten days in culture.

26. A population of cultured cells, derived from embryonic stem cells characterized by secretion of urea in an amount of no more than about 15 ng per 10⁶ cells per day after about ten days in culture.

27. A population of cultured cells, derived from embryoid bodies, wherein said population, after being cultured for at least about ten days, has cytochrome P450 activity corresponding to at least about 200 uM/ml resorufin after 30 minutes.

28. The population of cultured cells according to claim 16, produced by cultured in a collagen sandwich configuration and further supplemented with a morphogen.

29. The population of claim 28, wherein morphogen is SNAP or OSM.

30. A method of producing the population of cells according to claim 16, comprising:
a) providing a population of differentiated embryonic stem cells;
b) culturing said population of differentiated embryonic stem cells in a collagen sandwich configuration supplemented with a morphogen.

31. The method of claim 30, wherein said differentiated embryonic stem cells are present in a form of embryoid bodies.

32. The method of claim 31 further comprising a step of forming embryoid bodies from undifferentiated stem cells.

33. The method of claim 30, wherein the morphogen is OSM or SNAP.

34. The method of claim 33, wherein the morphogen is OSM, administered at a concentration of 10 ng/ml.

35. The method of claim 33, wherein the morphogen is SNAP, administered at a concentration of 250 uM.

36. A tissue construct for use in extracorporeal liver assist devices comprising at least a portion of the population of cells according to claim 16.

37. An implantable tissue construct comprising at least a portion of the population of cells according to claim 16.
